(19) United States
(12) Patent Application Publication
(10) Pub. No.: US 2018/0303345 A1
(43) Pub. Date: Oct. 25, 2018

(54) System and Method for Imaging Episodic Cardiac Conditions

(71) Applicant: CATHETER PRECISION, INC., Ledgewood, NJ (US)

(72) Inventor: Steve ADLER, Randolph, NJ (US)

(21) Appl. No.: 15/956,479

(22) Filed: Apr. 18, 2018

Related U.S. Application Data
(60) Provisional application No. 62/487,562, filed on Apr. 20, 2017.

(51) Int. Cl. A61B 5/00; A61B 5/0452; A61B 34/10

(52) U.S. Cl. CPC A61B 5/0044; A61B 5/055; A61B 34/10; A61B 5/0452

(57) ABSTRACT

Various embodiments provide a cardiac imaging system and method that includes using a portable electrocardiogram (ECG) recorder to record ECG data of a patient having an episodic cardiac condition for a time period sufficient for symptoms of the episodic cardiac condition to occur. The recorded ECG data may be provided to a processing unit along with other patient data. The processing unit may generate a three-dimensional (3D) activation map showing the propagation of electrical signals through the patient's heart. Based on the provided patient data, the processing unit may display an ablation point on the 3D activation map, the ablation point being configured to alleviate the episodic cardiac condition.

### CROSS-REFERENCE TO RELATED APPLICATION

This application claims the benefit of priority to U.S. Provisional Application No. 62/487,562, entitled “System and Method for Imaging Episodic Cardiac Conditions,” filed on Apr. 20, 2017, the entire contents of which are incorporated herein by reference.

### BACKGROUND

Some heart defects in the conduction system result in asynchronous contraction (arrhythmia) of the heart and are sometimes referred to as conduction disorders. As a result, the heart does not pump enough blood, which may ultimately lead to heart failure. Conduction disorders can have a variety of causes, including age, heart (muscle) damage, medications and genetics.

Premature Ventricular Contractions (PVCs) are abnormal or aberrant heart beats that start somewhere in the heart ventricles rather than a normal sinus beat that starts from the upper chambers of the heart. PVCs typically result in a lower cardiac output heart beat because the ventricles contract before they have had the chance to completely fill with blood, which may be symptomatic. PVCs may also trigger Ventricular Tachycardia (VT or V-Tach).

Ventricular tachycardia (VT or V-Tach) is a heart arrhythmia disorder caused by abnormal electrical signals in the heart ventricles. In VT, the abnormal electrical signals cause the heart to beat faster than normal, usually more than 100 beats per minute, with the beats starting in the heart ventricles.

VT generally occurs in people with underlying heart abnormalities. VT can sometimes occur in structurally normal hearts, and in these patients the origin can be in multiple locations in the heart. One common location is in the right ventricular outflow tract (RVOT), which is the route the blood flows from the right ventricle to the lungs. In other patients, such as those who have had a heart attack, scarring from the heart attack creates a milieu of intact heart muscle and a scar that predisposes patients to VT.

Catheter ablation is a treatment of choice in patients with VT and/or symptomatic PVCs. The targets for ablation are locations in the heart where the PVC's are occurring or locations where the onset of the VT is occurring. Currently, determining the proper ablation location may be problematic, because a patient may not exhibit symptoms during a brief testing session, such as testing of the patient in the doctor's office or during a catheterization procedure.

The common method of determining the proper location for ablation (for treating PVC or VT) is to conduct an electrophysiology (EP) study with internal mapping of the electrical activity of the heart. This EP study takes several hours and is only successful if the clinical (i.e., symptomatic) PVC or VT occurs and/or can be induced during the study.

Ambulatory monitoring (sometimes called Holter monitoring) is a clinical tool to record electro cardiogram (ECG) data while a patient is outside of the clinic or hospital setting. Ambulatory monitoring can be continuous in that the recording continues non-stop over a period of time, or can be episodic in that the recording only occurs when a specific episode(s) of interest occurs.

### SUMMARY

Various embodiments provide a cardiac imaging method, including using a portable electrocardiogram (ECG) recorder to record ECG data of a patient having an episodic cardiac condition for a time period sufficient for symptoms of the episodic cardiac condition to occur, and providing the patient ECG data to a processing unit that generates a three-dimensional (3D) activation map showing the propagation of electrical signals through the patient's heart. The 3D activation map may then be used by a processing unit to display an ablation point selected to alleviate the episodic cardiac condition.

In various embodiments, a cardiac imaging method may include recording electrocardiogram (ECG) data of a patient having an episodic cardiac condition using a portable ECG recorder for a time period sufficient for symptoms of the episodic cardiac condition to occur, providing patient data comprising the recorded ECG data to a processing unit, generating, by the processing unit, a three-dimensional (3D) activation map showing the propagation of electrical signals through the patient's heart based on the provided patient data, and displaying, by the processing unit, an ablation point on the 3D activation map, the ablation point being selected to alleviate the episodic cardiac condition. In some embodiments, the patient data provided to the processing unit may further include computed tomography (CT) or magnetic resonance imaging (MRI) data of the patient's heart and 3D image data of the patient's chest. In some embodiments, the time period may range from about 12 hours to about 48 hours. In some embodiments, the portable ECG recorder may include a 12-lead ECG recorder comprising 10 electrodes. In some embodiments, displaying the ablation point may include displaying multiple ablation points.

In some embodiments, recording ECG data using the portable ECG recorder may include receiving patient inputs on the portable ECG recorder identifying occurrences of the symptoms of the episodic condition, and recording the ECG data just prior to and during the patient inputs such that patient data includes primarily symptomatic ECG data.

In some embodiments, the ECG data may include symptomatic ECG data recorded during occurrence of symptoms of the episodic heart condition and non-symptomatic ECG data recorded at other times, and the method may further include using the processing unit to analyze the provided ECG data to identify symptomatic ECG data, and generating the three-dimensional (3D) activation map may include generating the 3D activation map based on the identified symptomatic ECG data and not the non-symptomatic ECG data.

In some embodiments, the ECG data may include symptomatic ECG data recorded during occurrence of symptoms of the episodic heart condition and non-symptomatic ECG data, and providing patient data including the recorded ECG data to the processing unit may include identifying the symptomatic ECG data, and providing to the processing unit patient data the symptomatic ECG data and excluding the non-symptomatic ECG data. In some embodiments, identifying the symptomatic ECG data may include analyzing the recorded ECG by the portable ECG recorder or by a physician.

In some embodiments, the episodic cardiac condition may include ventricular tachycardia (VT) or premature ventricular contraction (PVC). In some embodiments, displaying an ablation point may include displaying a location on the heart where the PVC occurs, or a location on the heart where the onset of the VT occurs.

Some embodiments may further include ablating the heart at the displayed ablation location. Such embodiments may further include generating an updated 3D activation map of the heart after the ablating of the heart. Such embodiments may further include determining, based on the updated 3D activation map, whether a desired synchronization of the heart was achieved.

Further embodiments include a cardiac imaging system, that includes a portable electrocardiogram (ECG) recorder configured to record ECG data of a patient having an episodic cardiac condition for a time period sufficient for symptoms of the episodic cardiac condition to occur, a display, and a processing unit coupled to the display that is configured to receive data from the portable ECG recorder, and configured with processor-executable instructions to perform operations of any of the methods summarized above. Such embodiments may further include

one or more of a computer tomography device, a magnetic resonance imaging device, a three-dimensional (3D) camera, an ECG recorder, a real-time imaging device, a synchronicity determining unit, and/or a virtual ablation point generator.

Further embodiments include a cardiac imaging system including means for recording ECG data of a patient having an episodic cardiac condition for a time period sufficient for symptoms of the episodic cardiac condition to occur, and means for performing functions of any of the methods summarized above.

Further embodiments include a non-transitory, processor-readable storage medium having stored thereon processor-executable instructions configured to cause a processor unit to use ECG data of a patient having an episodic cardiac condition recorded for a time period sufficient for symptoms of the episodic cardiac condition to perform operations of any of the methods summarized above.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is an example of a three-dimensional model of a heart.

FIG. 2A is a schematic diagram of a cardiac imaging system according to various embodiments.

FIG. 2B is a process flow diagram of a method of using the system of FIG. 2A according to various embodiments.

FIGS. 3A and 3B are plan views of 3D models of electrical activation of a heart according to various embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The term electrocardiogram (ECG) is used herein to refer to any method that (preferably non-invasively) correlates actual electrical activity of the heart muscle to measured or derived (electrical activity) of the heart. In case of a classical electrocardiogram, the differences in potential between electrodes on the body surface are correlated to the electrical activity of the heart. Derived ECG's can also be obtained in other ways, such as by measurements made by a so-called ICD (Implantable Cardioverter Defibrillator). In order to obtain such a functional image, an estimation of the movement of the electrical activity has to be provided.

Cardiac dyssynchrony and/or clinical PVC may have deleterious effects on cardiac function by depressing left ventricular (LV) mechanical performance, while increasing myocardial oxygen consumption. In addition, LV remodeling may also occur. Therefore, cardiac dyssynchrony and/or clinical PVC accelerate the progression of chronic congestive heart failure (CHF) and reduce patient survival.

During normal conduction, cardiac activation begins within both the left ventricular (LV) and right ventricular (RV) endocardium. In particular, electrical impulses (i.e., depolarization waves) travel substantially simultaneously through both the left and right ventricles.

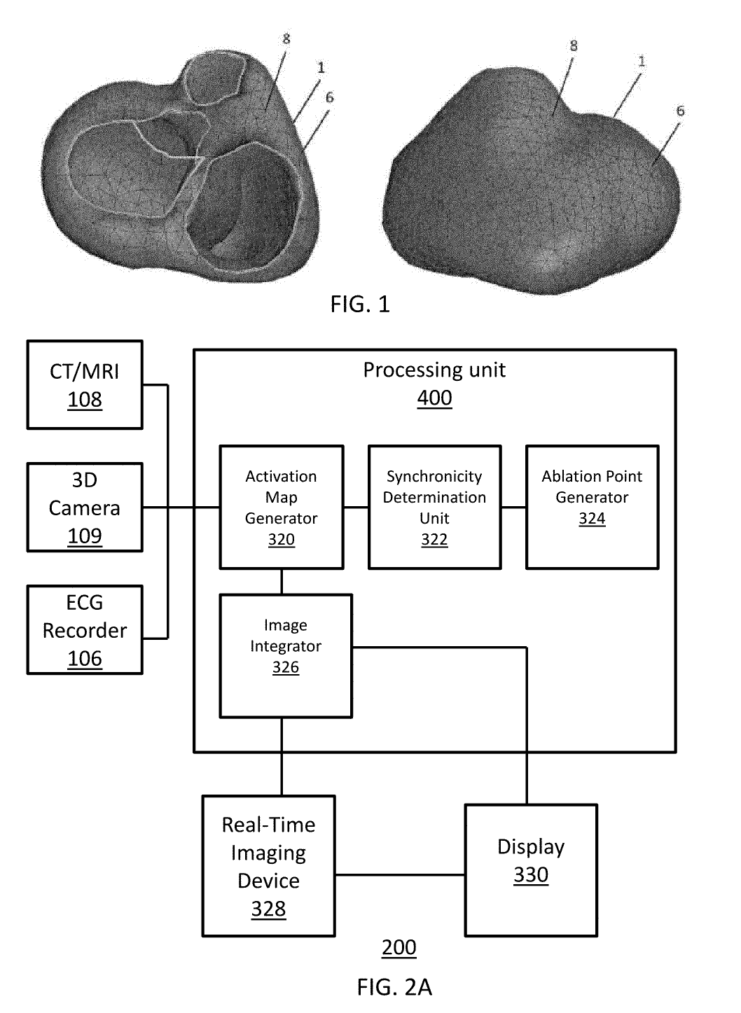

FIG. 1 shows a three-dimensional (3D) model of a heart 1 seen in two different directions. The 3D model includes a mesh 6 representing an outer surface of the heart, here the myocardial surface. In this example the 3D model also may include the septal wall. The mesh 6 has a plurality of nodes 8. In this example, the mesh 6 is a triangular mesh in which the surface of the heart is approximated by adjoining triangles.

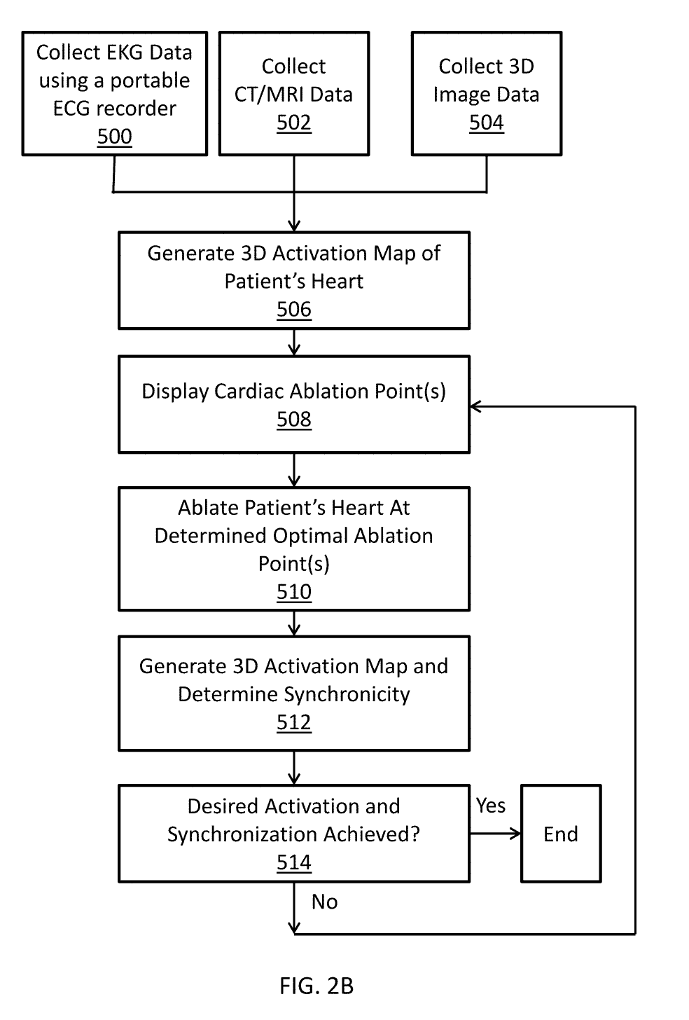

FIG. 2A is a block diagram of a cardiac imaging system 200 according to various embodiments. Referring to FIG. 2A, the imaging system 200 may include a processing unit 400, a CT or MRI device 108, a 3D camera 109, and an ECG recorder 106. The cardiac imaging system 200 may also include a real-time imaging device 328 and a display 330. The CT/MRI device 108 may be configured to generate a 3D model of the chest and/or heart of the patient. The 3D camera 109 may be configured to generate a 3D image of the patient's torso. The ECG recorder 106 may be configured to record ECD data from the patient, which may include extrinsic and/or intrinsic stimulation signals.

The processing unit 400 may include an activation map generator 320 configured to generate a 3D activation map of the heart of a patient based on patient data received from the CT/MRI device 108, 3D camera 109, and ECG recorder 106. The processing unit 400 may also include a synchronicity determining unit 322 configured to determine cardiac synchronicity, and a virtual ablation point generator 324 configured to identify ablation points selected to prevent the propagation of depolarization waves associated with PVC and/or VT.

The processing unit 400 may include an image integrator 326, which may be connected to a real-time imaging device 328, such as a fluoroscope, a radiography device, an X-ray computed tomography (CT) device, or the like. The image integrator 326 may compare and/or align the activation map and real-time images provided by the imaging device 328. Based on the comparison and/or alignment, the ablation point(s) may be added to the real-time images as virtual ablation point(s) to produce modified real-time images. The modified real-time images may be provided to and presented on a display 330.

In various embodiments, a workstation may be use that includes the processing unit 400, the display 330, and wired or wireless connections to other hardware, such as the CT/MRI device 108, the 3D camera 109, the ECG recorder 106, and/or the real-time imaging device 328. The workstation may also include an interface for controlling a surgical device, such as a catheter implantation device or other robotic surgical device.

Some patients may experience episodic VT and/or PVC, in which case the events or symptoms may not occur while a patient is tested at a hospital, during a catheterization procedure, or electrophysiology testing facility. To ensure sufficient ECG data is obtained for patients exhibiting episodic symptoms of VT and/or PVC, the ECG data may be recorded using a portable ECG recording device.

In various embodiments, the ECG recorder 106 may a portable ECG recording device, such as a Holter-type portable ECG recording device, that is capable of recording the patient's ECG data over an extended period of time. For example, the ECG recorder 106 may be 12-lead ECG recording device having 10 electrodes. Using a portable ECG recorder 106, ECG data may be collected over an extended period of time, which substantially increases the probability that the ECG data includes data recorded while symptoms of an episodic cardiac condition manifest. For example, a portable ECG recorder 106 may be used to collect ECG data over a time period ranging from about 8 to about 48 hours, such as from about 12 to about 36 hours, or about 24 hours. However, the ECG recorder 106 may be worn by a patient for any amount of time necessary to record ECG data during onset of a patient's episodic cardiac condition. Using a portable ECG recorder 106 to obtain readings over an extended period time increases the likelihood that the recorded ECG data will include symptomatic ECG data recorded during an episode of VT and/or PVC.

In some embodiments, the portable ECG recorder 106 may be configured to record all ECG data from a patient for a time period ranging from, for example, about 12 to about 48 hours. As such, the ECG data may include symptomatic and non-symptomatic ECG data. In some embodiments, the ECG recorder 106 may be configured to enable a patient to indicate when symptoms of an episodic cardiac condition occur, such as by pressing a button or touch screen when the patient feels a heart arrhythmia In response to such a patient indication, the ECG recorder 106 may be configured to identify corresponding symptomatic ECG data. For example, the ECG recorder 106 may be configured to tag, time stamp, or otherwise identify the symptomatic ECG data, so that the symptomatic ECG data may be distinguished from the non-symptomatic ECG data.

In some embodiments, the portable ECG recorder 106 may be used for a time period exceeding the recording capacity of the portable ECG recorder 106. In such situations, the portable ECG recorder 106 may be configured to temporarily record the most recent five or ten minutes of ECG data preceding the patient pressing a button or touch screen upon feeling symptoms, while overwriting older stored ECG data. If the patient detects symptoms of a cardiac event, the patient may instruct the ECG recorder 106 to store the relevant ECG data in a protected portion of the memory of the recording device. This may enable the portable ECG recorder 106 to record symptomatic ECG data without overflowing memory. In some embodiments, the symptomatic ECG data may be stored in a protected location, while non-symptomatic ECG data related to normal cardiac function may be stored in an unprotected location and periodically overwritten. As such, the memory requirements of the portable ECG recorder 106 may be reduced.

In some embodiments, the portable ECG recorder 106 may be configured to automatically detect a cardiac event and then store and protect the corresponding symptomatic ECG data. For example, the portable ECG recorder 106 may be configured to compare real-time ECG data to patterns of normal and/or abnormal ECG data and, based on the comparison, protect and store symptomatic ECG data while the real-time ECG data is consistent with an abnormal heart function. In some embodiments, the portable ECG recorder 106 may include an abnormal heart function detection algorithm to identify symptomatic ECG data that corresponds to abnormal heart function, such as VT and or PVC.

FIG. 2B is a process flow diagram illustrating a method of determining an ablation location using the system of FIG. 2A, according to various embodiments. With reference to FIGS. 2A and 2B, operations of the method may be performed using components and systems described with reference to FIG. 2A.

In operation 500, ECG data may be collected from a patient having an episodic abnormal cardiac function condition, such as VT and or PVC, using a portable ECG recorder 106. In operation 502, CT and/or MRI cardiac data may be collected from the patient. In operation 504, 3D image data of the patient's torso may be collected. Operations 500, 502, and 504 may occur in any order. Data recorded in operations 500, 502, and 504 may be collectively referred to herein as patient data. In some embodiments, a portable ECG recorder (e.g., 106) may be configured to output only identified symptomatic ECG data, such that non-symptomatic ECG data is excluded from the patient data.

In operation 506, a processing unit (e.g., 400) may receive the patient data, including the data collected in operations 500, 502, and 504, and use the data to generate a 3D activation map of the heart of the patient. In particular, the data may be provided to the activation map generator 320 of the processing unit 400. As noted above, the patient data may include only the symptomatic ECG data (e.g., clinical PVC ECG data). In such embodiments, the symptomatic ECG data may be used by the processing unit 400 to generate the activation map. For example, the processing unit 400 may be provided with only the tagged or otherwise identified ECG data of a cardiac event for generating the activation map. In some embodiments, a physician may review the recorded ECG data and select symptomatic portions that correspond to a cardiac event for use by the processing unit 400, when generating the activation map. In some embodiments, the processing unit 400 may be configured to filter the ECG data to identify symptomatic ECG data that corresponds to the onset of the episodic cardiac condition. For example, the processing unit 400 may be configured to use only tagged or otherwise identified symptomatic ECG data when generating the activation map. In some embodiments, the processing unit 400 may be configured to compare the received ECG data to stored ECG data indicating normal and/or abnormal heart function, to identify the symptomatic ECG data. In some embodiments, the processing unit 400 may include an abnormal heart function detection algorithm to identify the symptomatic ECG data from the received ECG data. The processing unit 400 may use only the identified symptomatic ECG data, in addition to the CT/MRI data and 3D image data, to generate the activation map in operation 506.

In operation 508, the processing unit 400 may identify and display one or more optimal cardiac ablation points on the activation map. For example, the activation map may be provided to the synchronicity determining unit 322 to determine cardiac synchronicity. This data may then be used by the virtual ablation point generator 324 to identify ablation point(s) corresponding to location(s) in the heart where the PVC's are occurring or cardiac locations where the onset of the VT is occurring. For example, one or more ablation points may be identified by determining locations of earliest ventricle activation. Ablation point(s) may then be added to the activation map and displayed in operation 508.

In some embodiments, the activation map and images generated by the real-time imaging device 328 may be provided to the image integrator 326. The image integrator 326 may compare and/or align the activation map and the real-time images. Based on the comparison and/or alignment, the ablation point(s) may be added to the real-time images as virtual ablation point(s) to produce modified real-time images. The modified real-time images may be provided to and presented on the display 330 in operation 508.

In some embodiments, the method may optionally include additional operations 510, 512, and 514. In operation 510, the activation map may be used to guide the positioning of a cardiac catheter to an ablation location and/or to guide diagnostic electrodes to appropriate locations on the heart, in real time. The patient's heart may then be ablated at the ablation location in operation 510.

In operation 512, an updated 3D activation map may be generated showing the results of the ablations performed in operation 510. For example, the updated activation map may be generated using ECG data collected after ablation is performed. Such ECG data may be collected during the procedure or afterwards, such as using a portable ECG recorder, such as a Holter-type ECG recorder, as in operation 500. The updated activation map may then be used to determine cardiac synchronicity.

In operation 514, a determination may be made regarding whether a desired synchronicity has been obtained. If so, the method may end. If not the processing unit 400 may return to operation 508 to display further cardiac ablation points as described. In some embodiments, this determination may be made by a clinician performing the procedure. In some embodiments, this determination may be made by the processing unit 400 and displayed to the clinician performing the procedure.

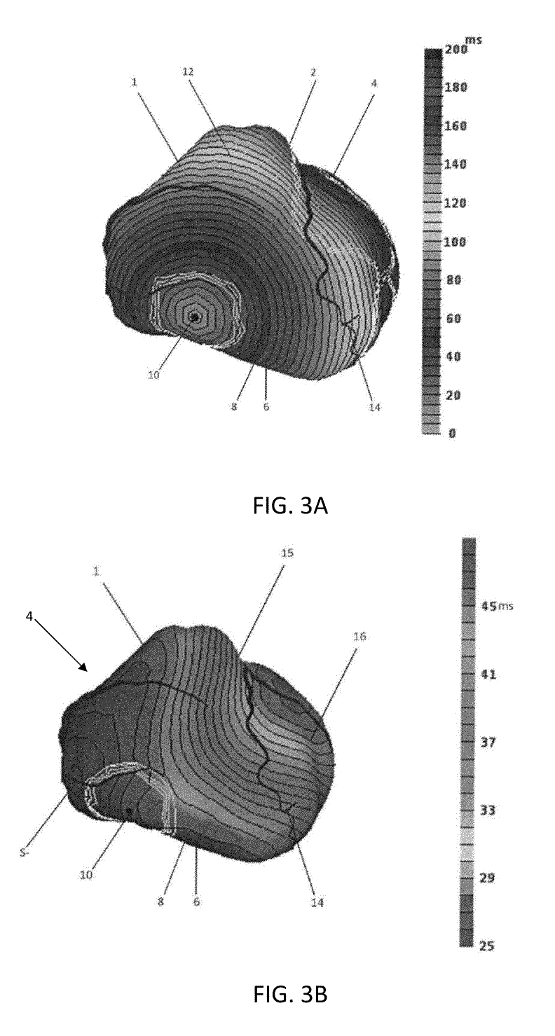

FIG. 3A shows a 3D model 4 illustrating the electrical activation of a heart 1. Such a 3D model may be generated by the system illustrated in FIG. 2A. In particular, FIG. 3A shows a ventricular surface of the myocardium with a septal wall 2. In general, the 3D model 4 may include a mesh 6 representing a ventricular surface of the heart, here an outer surface of the ventricular myocardium with septal wall as represented in FIG. 1. In the illustrated example, the mesh 6 has a plurality of nodes 8.

In the example illustrated in FIG. 3A, the heart 1 is stimulated beginning at an earliest activation location 10. From the earliest activation location 10, the electrical signals will travel through the heart tissue. Hence, different parts of the heart will be activated at different times. Each location on the heart has a particular delay relative to the initial stimulation. Each node 8 has associated therewith a value representative of a time delay between stimulation of the heart 1 at the earliest activation location 10 and activation of the heart at that respective node 8. In the example illustrated in FIG. 3A, locations that share the same delay time are connected by isochrones 12. As used herein, isochrones are lines drawn on a 3D heart surface model connecting points on this model at which the activation occurs or arrives at the same time. The delay time for nodes across the heart surface in this example is also displayed by differing rendering shades. The vertical bar indicates the time delay in milliseconds associated with the respective shade. It will be appreciated that the stimulation location 10 can be the location of intrinsic activation of the heart 1.

The 3D model 4 may also include further information. For example, the 3D model 4 may include cardiac blood vessels 14 and/or veins on the myocardium. This information may be added to the 3D model 4 in that nodes are indicated as being associated with such blood vessel. The blood vessels 14 may then be identified and optionally shown in the 3D model 4. Optionally, the processing unit 400 may include a first recognition unit arranged for automatically retrieving information representative of the location of such blood vessels from the patient's 3D anatomical model of the heart. The processing unit 400 may then automatically insert this information into the 3D model 4.

The 3D model 4 may also include information on scar tissue. Scar tissue locations may be obtained from delayed enhancement MRI images and added to the 3D model 4. Scar tissue may be simulated in the 3D model 4 by reducing the propagation velocity of electrical signals there through. Scar tissue may also be accounted for by selling the transition from one node to another to very slow or non-transitional for the areas in the heart wall where scar tissue is present. Optionally, the processing unit 400 may include a second recognition unit arranged for automatically retrieving information representative of the location of such scar tissue from the patient-specific three-dimensional anatomical model of the heart. The processing unit 400 may then automatically insert this information into the 3D model 4.

FIG. 3B shows the 3D model 4 illustrating simulated electrical activation of the heart 1 after ablation of the earliest activation location 10. In particular, the processor 400 may be configured to identify the earliest activation point 10 as an ablation point. The processor 400 may be configured to calculate changes to the electrical activation pattern of the heart 1, based on performing an ablation at the ablation point 10. In addition, the processor 400 may be configured to identify other ablation points S-, and calculate changes to the electrical activation pattern based on performing an ablation at such other points.

According to various embodiments of the present disclosure, the imaging systems described herein may be configured to determine cardiac synchronicity, as disclosed in U.S. Patent Application No. 2017/0011197, which is incorporated herein by reference in its entirety.

The foregoing method descriptions and the process flow diagrams are provided merely as illustrative examples and are not intended to require or imply that the operations of the various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the claims.

The hardware used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of receiver smart objects, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more aspects, the functions described may be implemented in hardware, software, firmware, or any combination thereof If implemented in software, the functions may be stored as one or more instructions or code on a non-transitory computer-readable storage medium or non-transitory processor-readable storage medium. The operations of a method or algorithm disclosed herein may be embodied in a processor-executable software module or processor-executable instructions, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include RAM, ROM, EEPROM, FLASH memory, CD-ROM or other optical disk storage, magnetic disk storage or other magnetic storage smart objects, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the claims. Thus, the present invention is not intended to be limited to the aspects and/or embodiments shown herein but is to be accorded the widest scope consistent with the following claims and the principles and novel features disclosed herein.

What is claimed is:

1. A cardiac imaging method, comprising:
recording electrocardiogram (ECG) data of a patient having an episodic cardiac condition using a portable ECG recorder for a time period sufficient for symptoms of the episodic cardiac condition to occur;
providing patient data comprising the recorded ECG data to a processing unit;
generating, by the processing unit, a three-dimensional (3D) activation map showing a propagation of electrical signals through the patient's heart based on the provided patient data; and
displaying, by the processing unit, an ablation point on the 3D activation map, the ablation point being selected to alleviate the episodic cardiac condition.

2. The method of claim 1, wherein the patient data provided to the processing unit further comprises computed tomography (CT) or magnetic resonance imaging (MRI) data of the patient's heart and 3D image data of the patient's chest.

3. The method of claim 1, wherein the time period ranges from about 12 hours to about 48 hours.

4. The method of claim 1, wherein the portable ECG recorder comprises a 12-lead ECG recorder comprising 10 electrodes.

5. The method of claim 1, wherein recording ECG data using the portable ECG recorder comprises:
receiving patient inputs on the portable ECG recorder identifying occurrences of the symptoms of the episodic condition; and
recording the ECG data just prior to and during the patient inputs such that patient data includes primarily symptomatic ECG data.

6. The method of claim 1, wherein:
the ECG data comprises symptomatic ECG data recorded during occurrence of symptoms of the episodic heart condition, and non-symptomatic ECG data recorded at other times;
the method further comprises using the processing unit to analyze the provided ECG data to identify symptomatic ECG data; and
generating the 3D activation map comprises generating the 3D activation map based on the identified symptomatic ECG data and not the non-symptomatic ECG data.

7. The method of claim 1, wherein:
the ECG data comprises symptomatic ECG data recorded during occurrence of symptoms of the episodic heart condition and non-symptomatic ECG data; and
providing patient data comprising the recorded ECG data to the processing unit comprises identifying the symptomatic ECG data, and providing to the processing unit patient data the symptomatic ECG data and excluding the non-symptomatic ECG data.

8. The method of claim 7, wherein identifying the symptomatic ECG data comprises analyzing the recorded ECG by the portable ECG recorder or by a physician.

9. The method of claim 1, wherein the episodic cardiac condition comprises ventricular tachycardia (VT) or premature ventricular contraction (PVC).

10. The method of claim 9, wherein displaying the ablation point comprises displaying a location on the heart where the PVC occurs, or a location on the heart where the onset of the VT occurs.

11. The method of claim 1, further comprising ablating the heart at the displayed ablation location.

12. The method of claim 11, further comprising generating an updated 3D activation map of the heart after ablating the heart.

13. The method of claim 12, further comprising determining, based on the updated 3D activation map, whether a desired synchronization of the heart was achieved.

14. The method of claim 1, wherein displaying the ablation point comprises displaying multiple ablation points.

15. A cardiac imaging system, comprising:
a portable electrocardiogram (ECG) recorder configured to record ECG data of a patient having an episodic cardiac condition for a time period sufficient for symptoms of the episodic cardiac condition to occur;
a display; and
a processing unit coupled to the display, configured to receive data from the portable ECG recorder, and configured with processor-executable instructions to perform operations comprising:
receiving patient data comprising ECG data recorded by the ECG recorder;
generating a three-dimensional (3D) activation map showing a propagation of electrical signals through the patient's heart based on the provided patient data;
using the 3D activation map to select one or more ablation points to alleviate the episodic cardiac condition; and
displaying on the display the selected one or more ablation points on the 3D activation map.

16. The imaging system of claim 15, further comprising one or more of:
a computer tomography device;
a magnetic resonance imaging device;
a three-dimensional (3D) camera;
an ECG recorder;
a real-time imaging device;
a synchronicity determining unit; and
virtual ablation point generator.

17. The cardiac imaging system of claim 15, wherein the portable ECG recorder comprises a 12-lead ECG recorder comprising 10 electrodes.

18. The cardiac imaging system of claim 15, wherein the portable ECG recorder is further configured to:
receive patient inputs identifying occurrences of the symptoms of the episodic condition; and
record the ECG data just prior to and during the patient inputs such that the patient data includes primarily symptomatic ECG data.

19. The cardiac imaging system of claim 15, wherein:
the ECG data recorded by the portable ECG recorder comprises symptomatic ECG data recorded during occurrence of symptoms of the episodic heart condition, and non-symptomatic ECG data recorded at other times;
the processing unit is further configured with processor-executable instructions to perform operations comprising using the processing unit to analyze the provided ECG data to identify symptomatic ECG data; and
the processing unit is further configured with processor-executable instructions to perform operations such that generating the 3D activation map comprises generating the 3D activation map based on the identified symptomatic ECG data and not the non-symptomatic ECG data.

20. The cardiac imaging system of claim 15, wherein the processing unit is further configured with processor-executable instructions to perform operations such that displaying the ablation point comprises displaying a location on the heart where premature ventricular contraction (PVC) occurs or a location on the heart where the onset of ventricular tachycardia (VT) occurs.

21. The cardiac imaging system of claim 15, the processing unit is further configured with processor-executable instructions to perform operations further comprising:
generating an updated 3D activation map of the heart after an ablation of the ablation point is performed; and
determining, based on the updated 3D activation map, whether a desired synchronization of the heart was achieved.

22. A cardiac imaging system, comprising:
means for recording electrocardiogram (ECG) data of a patient having an episodic cardiac condition for a time period sufficient for symptoms of the episodic cardiac condition to occur; and
means for receiving patient data comprising ECG data recorded by the ECG recorder;
means for generating a three-dimensional (3D) activation map showing a propagation of electrical signals through the patient's heart based on the provided patient data;
means for using the 3D activation map to select one or more ablation points to alleviate the episodic cardiac condition; and
means for displaying the selected one or more ablation points on the 3D activation map.

23. A non-transitory, processor-readable storage medium having stored thereon processor-executable instructions configured to cause a processor unit of a cardiac imaging system to perform operations comprising:
receiving patient data comprising electrocardiogram (ECG) data recorded by a ECG recorder for a time period sufficient for symptoms of the episodic cardiac condition to occur in a patient's heart;
generating a three-dimensional (3D) activation map showing a propagation of electrical signals through the patient's heart based on the provided patient data;
using the 3D activation map to select one or more ablation points to alleviate the episodic cardiac condition; and
displaying on the display the selected one or more ablation points on the 3D activation map.
